(19) United States
(12) Patent Application Publication
(10) Pub. No.: US 2004/0209954 A1
(43) Pub. Date: Oct. 21, 2004

(54) Compositions and methods for treating body malodor and fungal overgrowth in mammals

(76) Inventor: Alison B. Lukacsko, West Windsor, NJ (US)

Correspondence Address: Vic Lin, MYERS DAWES ANDRAS & SHERMAN, LLP, Suite 1150, 19900 MacArthur Blvd., Irvine, CA, 92612, US

(21) Appl. No.: 10/826,238

(22) Filed: Apr. 16, 2004

Related U.S. Application Data
(60) Provisional application No. 60/464,079, filed on Apr. 18, 2003.
(60) Provisional application No. 60/469,434, filed on May 9, 2003.

(51) Int. Cl. A61K 31/205; A61K 31/14

(52) U.S. Cl. 514/554; 514/642

(57) ABSTRACT

The present invention is concerned with antimicrobial compositions suitable for bacteriostatic/bacteriocidal and fungistatic/fungicidal applications. Anticholinergic quaternary amine compounds are not known for their activity as antimicrobial agents, but have been determined as presenting a substantial benefit while exhibiting significantly reduced toxic effects over conventional treatments. Anticholinergic quaternary amine compounds are incorporated in an excipient matrix, at concentrations of from about 0.001% to about 10.0% (within an order of magnitude) either alone or in combination with conventional antifungal/antibacteriological agents. Anticholinergic quaternary amine compounds have antimicrobial pharmaceutical efficacy as well as the ability to enhance, accelerate and assist antimicrobial activity of other conventional preparations. Methods of use include suitability for body deodorization compositions, anti fungal compounds and treatments for common microbial infections and physiological manifestations of bacterial colonization, such as body malodor.

### RELATED APPLICATIONS

Pursuant to 35 USC § 119, the present application relates to and claims priority from U.S. Provisional Patent Application Serial No. 60/464,079, filed on Apr. 18, 2003, and U.S. Provisional Patent Application Serial No. 60/469,434 filed on May 9, 2003, both of which are incorporated herein by reference.

### FIELD OF THE INVENTION

The present invention is directed to broad spectrum antimicrobial compositions and, more particularly to antibacterial and antifungal compositions and compositions and methods for prevention and treatment of pathogenic and non pathogenic conditions killing or inhibiting growth of bacteria or fungi, and obtaining physiological/psychological effects included with an antimicrobial deodorization benefit.

### BACKGROUND OF THE INVENTION

The present invention relates to novel methods for the treatment and prevention of certain conditions by killing or inhibiting growth of fungi and/or bacteria with an anticholinergic quaternary ammonium compound (ACQA) as well as antimicrobial and antifungal compositions or devices for use in mammals.

Specifically, human beings are natural hosts for a diversity of microorganisms. Common sites of colonization include the oral and vaginal cavities, the soles or interdigital areas of the foot, intertrigineous zones, as well as the gastrointestinal tract. Under certain conditions, when the local ideal homeostatic balance is disturbed, such organisms are able to create problems related to health and/or hygiene of a human being. This may be referred to as an infection in a colloquial way, but it need not be pathogenic to fall within the scope of this invention. Skin infections and localized malodor are but two examples of such problems. According to an estimate developed by the United States National Health Survey of 1971-1974, the prevalence of all cutaneous infections (mycoses) is put at about 88 out of 1,000 persons. Often, the offending microorganism lives commonly on the normal skin of healthy individuals and, as such, mycotic disease is not considered contagious.

Nevertheless, and in the particular case of fungal infections, such opportunistic infections, also termed mycoses, can be characterized by the extent to which they penetrate affected tissue. In this particular schema, mycoses can be superficial, meaning that they affect only the surface of the skin. Alternatively, mycosis may be a dermatophyte infection, in which case it has the capability to infect all cornified components of the skin, including the hair and nails. In the case of dermatophytes, invasion of the viable epidermis and vascular dermis is unusual. Additionally, a third type of infection is termed a candida infection, so called because species of Candida are the causative organisms. Candida has the greatest pathogenic potential among the mycoses described above. Candidiasis is a general term for a variety of local and systemic processes caused by colonization or infection of a host by a certain species of the Candida yeast. Fungal infection can be systemic, meaning that the causative organisms circulate in the blood and/or effect internal body organs and systems. Immunocompromised individuals, such as those with HIV/AIDS or undergoing chemotherapy, frequently exhibit fungal infections. In those situations, fungal infections may be life threatening.

Superficial, dermatophyte and candida infections or conditions are, in general, noninvasive. This noninvasive characteristic distinguishes them from the more pathogenic fungi referred to above. Such noninvasive, cutaneous infections are often referred to by the term “tinea,” except for those caused by Candida, which are termed candidiasis. Candidiasis occurs worldwide. Superficial infections of the skin, mouth and other mucus membranes are also universal. Clinically, Candidiasis manifests as superficial mucocutaneous infections, chronic mucocutaneous candidiasis, or systemic infection. Superficial mucocutaneous infections can occur in any area of either the skin or a mucus membrane. Invasive systemic disease has become a problem due to the use of high doses of antibiotics that interfere with or destroy normal bacterial flora, immunosuppressive agents and agents toxic to bone marrow, e.g., during cancer related chemotherapy.

Neutropenia is a major risk factor for Candida dissemination. Candidiasis is also seen among immunocompromised individuals such as AIDS patients, organ transplant recipients, patients receiving parenteral nutrition, and cancer patients undergoing radiation treatment and/or chemotherapy. It is the most common opportunistic mycosis internationally. The most common etiologic agent is Candida Albicans. Other infectious species include C. tropicalis, C. parapsilosis, C. stellatoidea, C. kusei, C. parakawsei, C. lusitanine, C. pseudotropicalis, C. guilliermondi and C. glabrata. Candida Albicans is normally found in the mouth, throat, gastrointestinal tract and vaginal areas of human beings. Non-albicans species frequently colonize the skin.

A Latin term, according to the anatomic location is used in combination with the “tinea” descriptor to define specific mycoses. Capitius and Pedis, for example refer to the head and foot, respectively. Tinea Versicolor deviates from this general rule but nonetheless is another of the common mycotic infections. Under certain conditions, the cutaneous microenvironment might change, facilitating proliferation of the organism. Such is the case for Tinea Versicolor and Tinea Nigra, for example. Similarly, in the presence of excessive moisture, such as might occur with sweaty feet, fungi is able to proliferate and create an infection such as Tinea Pedis, more commonly referred to as athletes foot.

In contrast to fungi that produce superficial conditions, those associated with dermatophyte infection have a capability to infect cornified components of the skin including hair and nails. Dermatophytosis is a chronic fungal infection of the skin, hair or nails by dermatophytes, which include members of the species trichophyton, microsporum and epidermophyton. Infection of the foot (tinea pedis), scalp (tinea capitis) are most common, although widespread infection on non-hair bearing skin (tinea corporis) also occurs. Clinical manifestations vary and may be present within or on the skin as fissuring or lesions with scaling, vesicles or pustules, along with alopecia on the scalp, or on the nails as discolored or chalky, crumbling nails. Both topical and systemic therapies may be used to treat dermatophyte infection or conditions, including topically administered imidazoles and triazoles, such as but not limited to itraconazole, miconazole, ketoconazole and econazole, haloprogin, undecylic acid, ciclopirox olamine, tolnaftate and terbinafine. Although there are numerous dermatophytes, seven species of dermatophytes cause more than 90% of all infections. Additionally, there are about seven clinical anatomic infection syndromes, with Tinea Pedis being the most common, occurring approximately three times more frequently than Tinea Ungulum, the next most frequent.

Amongst the general public, fungal infections such as those referred to above are not considered life threatening. This is in contrast to the effects of systemic infections among immunocompromised individuals. However, even in otherwise healthy individuals, fungal infections or conditions can be quite bothersome, producing both physical and psychological discomfort. Additionally, certain infections such as Tinea Ungulum (infection of the nails) are not easily treatable, leaving a patient to live with unsightly disfigurement including deformed, discolored, thickened and opacified nails, perhaps for the remainder of their lifetime. This can be quite psychologically disabling to individuals whose hands are in the public view. Thus, although not life threatening, cutaneous mycoses can negatively affect the quality of life for sufferers. This is perhaps why approximately 7% of all outpatient visits to large medical clinics across five continents, was for treatment of cutaneous fungal infections.

Fungal infections are becoming a major health concern for a number of reasons. Certain of these reasons relate to the limited number of anti-fungal agents available, along with the increasing incidence of species resistant to older anti-fungal agents. In this regard, the majority of known anti-fungal agents fall into one of three main categories. The major category includes polyene derivatives, including amphotericin B and structurally related compounds nystatin and pimaricin, which are only administered intravenously. Specifically, these compounds are broad-spectrum anti-fungals that bind to ergosterol, a component of fungal cell membranes, in order to disrupt fungal cell membranes, thereby leading to fungal cell death. Amphotericin B, administered by the intravenous route, is usually effective for systemic mycoses. The utility of amphotericin B is limited by certain toxicological side effects, such as kidney damage, fever, anemia, low blood pressure, headache, nausea, vomiting and phlebitis. The unrelated anti-fungal agent flucytosine (5-fluorocytosine), an orally absorbed drug, is frequently used as an adjunct to Amphotericin B treatment for some forms of Candidiasis and cryptococcal meningitis. Its adverse affects include bone marrow depression with leukopenia and thrombocytopenia. Another anti-fungal agent is griseofulvin, a fungistatic agent that is administered orally for fungal infections of skin, hair or nails that have not responded to topical treatment. Even more recently, systemic, tablet based formulations have been brought to market, as exemplified by the commercially available product “Diflucan.”

An additional major group of anti-fungal agents includes azole derivatives, which impair synthesis of ergosterol, and which lead to accumulation of metabolites that disrupt the function of fungal-membrane bound enzyme systems (e.g., cytochrome P450), thereby inhibiting fungal growth. Significant inhibition of mammalian P450 results in important drug interactions. This group of agents includes such compounds as ketoconazole, clotrimazole, miconazole, econazole, butoconazole, oxiconazole, sulconazole, terconazole, fluconazole and itraconazole.

These agents may be administered to treat systemic mycoses. Ketoconazole, an orally administered imidazole, is used to treat nonmeningeal blastomycosis, histoplasmosis, coccidioidomycosis and paracoccidioidomycosis in non-immunocompromised patients, and is also useful for oral and esophageal Candidiasis. Adverse effects include rare drug induced hepatitis. Ketoconazole is also contraindicated in pregnancy. Itraconazole appears to have fewer side effects than Ketoconazole and is used for most of the same indications. Fluconazole also has fewer side effects than Ketoconazole and is used for oral and esophageal candidiasis and cryptococcal meningitis. Miconazole is a parenteral imidazole with efficacy in coccidioidomycosis and several other mycoses, but has side effects including hyperlipidemia and hyponatremia. The azole derivatives are commonly used via the topical route to treat superficial and dermatophyte infections, in which situations their safety profile is considerably more favorable. Nevertheless, they do present the risk of higher order safety issues if significant absorption occurs.

Beyond symptomatic relief, the current treatment paradigm for active cutaneous mycoses consists mainly of the use of azole or alyamine-based anti-fungal drugs in either topical, parenteral or oral form. In contrast to older drugs, such as Haloprogin, Nystatin, and Amphoterachin B, the allyamine and azole-type drugs exhibit broad-spectrum actions with positive effects against a range of dermatophytes and candida. Depending on which mycosis is being treated, resolution of the infection can take anywhere from two weeks to many months. The ally amine creams generally require a shorter treatment course (approximately 1-4 weeks) than do azoles (approximately 2-8 weeks), but both medication groups produce similar cure rates. When the infection is severe, supplementation of localized topical treatment with an oral medication may be required.

Resistance to anti-fungal agents has become more apparent in recent years and may worsen with the increase in prophylactic therapy. Moreover, treatment of fungal infections has lagged behind bacterial chemotherapy, and not unsurprisingly, there are substantially fewer anti-fungal drugs than anti-bacterial drugs. There is intense interest in identifying new drugs with different modes of action against fungal infections. The current repertoire of anti-fungals has limitations, such as insufficient efficacy, the need for intravenous administration, high cost, serious side effects, and the appearance of resistant fungal strains. Importantly, most of the current treatments are only fungistatic, meaning that they inhibit fungal growth but do not cause outright fungal death. Subsequent clearing of these inhibited fungi is inadequate in patients with defective immune systems. Thus, it is imperative to identify fungicidal agents and, if possible, their cellular targets that, when impaired, lead to fungal cell death.

In another aspect, fungi may be viewed as merely one undesirable form of the various microorganisms that reside on the human body; another might be a bacterium. Like fungi, bacteria may be found on or within the skin structure. Bacterial infections or conditions may be associated with cutaneous abscesses, deeper cellulitis and infections or wound infections. Beyond infections of bacterial orogin, bacteria on the surface of the skin are known to produce, promote or excrete aromatic organics associated with body malodor, which can create problems related to health and/or hygiene of a person. One such condition is termed bromhydrosis or smelly sweat,

Such organisms on skin are nourished by various skin-secreted substances, skin cell debris, breakdown products of the skin and breakdown products of the organisms themselves. The “skin secretions” are eccrine and apocrine sweat, and lipid-soluble sebum. Under moist conditions, such as sweaty feet for example, the skin itself degrades and develops a malodor when the organic residue is acted upon by resident organisms. Eccrine sweat, which is secreted by eccrine sweat glands, is normally odorless and remains odorless after secretion, although odoriferous food and drug substances may be excreted with it. Apocrine glands are normally associated with hair follicles and are confined mainly to the groin, perianal, areola and armpits. They produce a scanty, milky substance that is odorless upon secretion, but becomes odoriferous upon bacterial decomposition. Apocrine glands are considered to be a primary contributor for the malodor generally referred to as body odor or “BO.” The sebaceous glands are distributed over the skin surface except the palms and soles dorsae. They are most numerous on the scalp, forehead, face, back and chest. The secretion, sebum, consists mainly of fatty materials, wax esters, cholesterol and its esters and squalene. Sebum is typically associated with acne.

Specifically, body odor is most commonly caused by fatty acids on skin and from malodors from bacterial sources. The unpleasant odors are mainly organic molecules which have different structures and functional groups, such as amines, acids, alcohols, aldehydes, ketones, phenolics, polycyclics, indoles, aromatics, polyaromatics, etc. They can also be made up of sulfur-containing functional groups, such as, thiol, mercaptan, sulfide and/or disulfide groups.

Numerous approaches to reducing body odor have been tried, but without significant long-term success. Strategies include odor masking, odor neutralizing, odor quenching, inhibition of enzymes that lead to odor formation (esterases, for example), killing or damaging the bacteria which make the odor or creating an environment which is hostile to those bacteria such as drying the environment so as to retard the bacterial growth. As regards foot odor, for example, powders may be used to absorb moisture and prevent degradation of the skin. Alternatively, for example, the odors may be covered up with fragrances; numerous attempts have been made to conceal body odors through the use of perfumes. Not only are such perfumes often inadequate at fully concealing the body odors, but they are also very often irritating to the user's skin. Additionally, the perfume odor itself may be irritating or offensive to the user's respiratory system and/or olfactory senses, as well as to nearby individuals.

In a different approach, the malodor causing material may be chemically neutralized or reacted via oxidation, the odor may be functionally adsorbed or absorbed or quenched, for example, with activated charcoal, with cyclodextrins or zeolites. Zeolites such as those marketed under the trade name ABSCENTS by the Union Carbide Corporation and UOP are known odor absorbers. However these commonly known solid odor absorbers, in addition to known activated charcoal odor absorbers, lose functionality when wet. Therefore, when wetted by body fluids or when carried in an aqueous solution, these odor absorbers are not preferred as they lose their desired odor absorbent characteristics. Furthermore, zeolites can cause a “harsh” feel if too much is deposited onto the skin. Reducing the vapor pressure is a viable means to reduce perception of the odor. Yet another attempt at controlling body odor is found in U.S. Pat. No. 4,382,079, to Marschner, issued May 3, 1983, which discloses the use of sodium bicarbonate as an underarm deodorant to neutralize offending body odor. Blockers of odor perception have, likewise, been attempted. Zinc salts of ricinoleic acid bind odor molecules and can quench odors; however, they can interact unfavorably with perfume components. Zinc glycinate and triethylcitrate have been explored as esterase inhibitors; materials which can inhibit enzyme activity. Glyceryl and sucrose fatty acid esters have been reported in the literature as deodorizers as has been glycerolether. New concepts for controlling mammalian body malodor associated with microbial activity include other absorbents such as chitosan, newer bacterial enzyme inhibitors, odor inhibiting precursors and compounds that impair bacterial adhesion onto skin.

Odor causing bacteria and fungi often flourish in warm, moist conditions; particularly where they have easy access to nourishment such as skin secretions and skin cell debris. As an alternative means of preventing microbial-related odor formation, attempts are made to deprive the organisms responsible for the body odor of the moist/humid environment they need to proliferate and grow. Thus, attempts have been made to control odor through moisture absorption. As an alternative, antiperspirants act by suppressing the manufacture of sweat, or by blocking the expression of sweat, generally eccrine in nature, onto the skin surface. Because the microorganisms that generally produce malodor live optimally in the presence of moisture, the reduction of the surface moisture by virtue of reduced sweat output may carry indirect deodorizing benefits as well. Indeed, some antiperspirant materials, such as the metal salt antiperspirants in common use today for example, are said to have deodorant properties because of their adverse effect on the bacterial environment.

However, it is generally recognized that this is not their primary mechanism of action, thus deodorization cannot be assumed. Notwithstanding the antibacterial properties of the metal salt antiperspirants, one might envision the utility of antiperspirants with unique mechanisms of action, e.g. neurophysiologic or physical blockage as from occlusion or film formation and such products would carry an inherent risk of background malodor development. Such malodor might, for example, be a risk if there is breakthrough sweat, i.e., sweating despite the presence of the antiperspirant.

Each of the above strategies for altering odor formation has significant limitations. Powders and powder-based compositions of the prior art may be difficult to apply and have limited absorption capabilities. Some commonly used powders, such as cornstarch, actually enhance growth of microorganisms and have the potential to increase odor development. Therefore, use of the body powders of the prior art is undesirable and/or ineffective for day-to-day body odor control for the entire body. Conventional antiperspirants are not useful in a body odor control product for use over the entire body as such salts may be irritating to the skin of a large number of users, particularly when applying them to sensitive areas such as the pelvic region and they frequently damage fabric and leave unacceptable white residue on skin and clothing.

Numerous other deodorant compositions aimed at combating odor associated with the skin secretions have been described in the chemical and cosmetic literature. These generally are in the form of emulsion sticks or suspension sticks, but also may be powders, aerosols, roll-ons, pads, pump sprays, and even soap bars. As described above by means of general, but not exhaustive illustration, known deodorants attempt to control odor through a variety of means. In addition to the above, deodorants may include antimicrobial compounds that help destroy and/or control the amount of bacteria present on the skin, thereby minimizing odor produced via bacterial metabolism of the skin secretions. U.S. Pat. No. 5,525,331, to Betts, issued Jun. 11, 1996, discloses compositions that inhibit the growth of microorganisms in the body-secretions. Indeed, many commercial cosmetic deodorant products are designed to mitigate the development of unpleasant malodors by killing the bacteria that create them. For optimal utility, activity against a wide range of organisms, both bacterial and fungal is advantageous. Triclosan, a broad-spectrum anti-microbial agent is an example of a compound in current use that exhibits some of these properties. As a result of its activity, it has found increasingly popular use in personal care and household products including toothpaste, deodorant soaps, deodorants, antiperspirants, body washes, detergents, dish washing liquids, cosmetics, antimicrobial creams, lotions and hand soaps. It is also an additive in plastics, polymers and textiles to impart antibacterial properties.

Triclosan, however, has issues associated with its use. Triclosan is a chlorophenol, a class of chemicals suspected of causing cancer in humans. It is principally bacteriostatic with relatively minor fungistatic activity. By virtue of its chemical structure (polychlorophenoxyphenol), triclosan can become contaminated with potentially toxic synthetic impurities (dioxins). In addition, triclosan has very limited water solubility. This attribute greatly limits formulation flexibility and requires the use of organic solvents and possibly irritating surfactants to enhance the biological activity.

Beyond triclosan, there are other antimicrobial agents that have been used as topical antiseptics. These include ethanol, methanol, thimerosol, phenolic compounds, cresols, resorcinol compounds and even hydrogen peroxide. Relative potency, long-term safety, irritation, and odor are among the characteristics of these materials that limit their use.

There continues to exist a need in the art for new products and methods for use as anti-microbial agents. Products and methods responsive to this need would ideally involve substantially non-toxic compounds available in large quantities. Ideal compounds would have a rapid effect and a broad spectrum of fungicidal or fungistatic as well as bactericidal or bacteriostatic activity against a variety of different microbial species. Even if an agent were not effective on its own, when administered in conjunction with other anti-fungal or anti-bacterial agents, the combination may be more effective or the combination may permit a reduction in the amount of the additional anti-fungal or anti-bacterial agent. This reduction may beneficially limit potential toxic responses or reduce the high cost of treatment.

One having skill in the art will understand that there has been much effort directed toward identifying effective, safe, convenient antimicrobial compounds for use in commercial products. Quaternary ammonium compounds are cationic surfactants that have nonspecific activity against gram positive and gram-negative bacteria associated with their chemical toxicity. These materials have cleansing properties, emulsify sebum and have a detergent effect to remove dirt, bacteria and desquamated epithelial cells. Their antiseptic value stems largely from cleaning away bacteria and contaminating dirt and debris. Mechanistically, quaternary ammonium compounds disrupt the cellular membranes and denature lipoproteins of microbes. Cationic agents, such as Benzalkonium chloride, benzethonium chloride and methylbenzethonium chloride, have been proposed by the USFDA as topical ant infective agents for OTC use. It is notable that these compounds, if used undiluted, may cause serious irritation. Accordingly, useful concentrations will vary from about 1:200,000 on broken or diseased skin to about 1:750 on intact skin. Also notable, is that these particular “xxxonium chloride” compounds are not suitable for any other uses, and in particular, are not deemed suitable for other pharmacological applications.

The current invention relates to a unique method for treating fungi and bacteria and the conditions they may create, thereby clearing the condition, deodorizing, reducing the formation of, and/or preventing the formation of body odor comprising the application of an antimicrobial composition or device containing a quaternary amine compound which functions otherwise as an anticholinergic drug (ACQA).

ACQAs refer to a group of drugs containing a quaternary amine structure and which have been used classically to block the physiological affects of acetylcholine (ACH) on its receptor. Owing to the pharmacology of ACH, these drugs have found clinical use in pre-anesthetic cocktails (parenteral), for the reduction of gastric acid secretion (oral), in rhinitis (intranasal, oral) and intraocularly as a mydriatic delivered via eye drops. Glycopyrrolate (GP), an example of a compound in this class, was approved for use over thirty years ago. In general, amines are generally recognized as compounds that contain one or more organic groups bonded to nitrogen. Amines are classified as primary, secondary, tertiary and quaternary according to how many organic groups are bonded to the nitrogen atom. Since each amine nitrogen has a lone pair of electrons, when a fourth organic group bonds to the nitrogen through the remaining lone pair, the product is a quaternary ammonium ion, which has a positive charge and which forms ionic compounds.

Classically, there may be significant adverse side effects with anti-cholinergic drugs. These adverse side effects might include dry mouth, increased heart rate, urinary retention and CNS depression. These are, however, minimized in ACQA's given the quaternary amine structure of these compounds. Being charged at physiologic pH means that these compounds do not cross cell membranes easily. For that reason, with systemic use, adverse effects are generally absent and/or mild. Thus, drugs such as glycopyrrolate have been used as chronic treatments for a diversity of conditions.

In one aspect, an antimicrobial composition is provided for topical application to control odor formation associated with mammalian fungal and/or bacterial infestations or infections. The composition comprises a quaternary amine recognized otherwise as having anticholinergic properties in a weight per volume between 0.001% and 10% of the composition in combination with an excipient or a delivery vehicle.

### SUMMARY OF THE INVENTION

The present invention is directed to a broad spectrum antimicrobial (bacteriostatic or bactericidal or fungistatic or fungicidal) composition or device comprising, in a general form, an excipient material suitable for topical or systemic administration and a quaternary amine compound having anticholinergic activity. For ease of reference herein, we may refer generally to the anticholinergic quaternary amine (ACQA) as antimicrobial. The concentration of the anticholinergic quaternary amine compound in the composition is in an amount of from about 0.0001% to about 20% w/w, and preferably in an amount of from about 0.001% to about 10% w/w. More preferably, the concentration of the anticholinergic quaternary amine compound in the composition is in an amount of from about 0.001% to about 5% w/w.

The broad spectrum ACQA antimicrobial composition may further comprise a non-ACQA anti-fungal or anti-bacterial agent having a recommended concentration defining an effective therapeutic dose and wherein the recommended concentration of the non-ACQA agent is substantially reduced without reduction in effective therapeutic effect by combination of the non-ACQA anti-fungal agent with the anticholinergic quaternary amine compound. Advantageously, the antifungal and/or antibacterial effect of the combination is accelerated and enhanced over the antimicrobial effect of the non-ACQA anti-fungal agent alone. Alternatively, in combination, the therapeutic effect is broadened without negative effects relating to safety.

In one aspect of the invention, the anticholinergic quaternary amine compound comprises glycopyrrolate. The non-ACQA anti-fungal agent comprises an imidazole or triazole compound, or any other therapeutically effective anti-fungal agent chemically compatible with a selected anticholinergic quaternary amine compound. This might include, for example, a peptide with antimicrobial activity. In another aspect, the invention is directed to a method for treating a fungal infection or condition comprising the steps of preparing a therapeutically effective amount of an anticholinergic quaternary amine compound and administering or delivering said anticholinergic quaternary amine compound to an area of a human body exhibiting said fungal infection. The administration step comprises contacting a fungi residing on or within the affected area with said anticholinergic quaternary amine compound or treating the fungal condition systemically.

The method according to the invention further comprises administration of the anticholinergic quaternary amine (ACQA) compound as a formulation in conjunction with a non-physiologically active base or support material. The non physiologically active base or support material includes additional components having non-fungal or nonbacterial activities, such as excipient components, the additional components selected from the group consisting of keratolytics, vitamins, antioxidants, anti-inflammatories, humectants, antihistamines, antiperspirants, antibacterials, moisturizers, lubricants, UV-protectants, astringents, antiseptics, skin calming agents, growth factors, wound healing enhancers, depilatories, skin or hair cleansers or conditioners, and hair growth retardants or enhancers.

A particular advantage of the present invention lies in its ability to support both topical and systemic administration. The administration step includes topical application of the anticholinergic quaternary amine compound as a preparation selected from the group consisting of patches, films sticks, gels, aerosols, non-aerosol sprays, solutions creams, ointments, lotions, mousses, powders, soft solids, and roll-ons. The administration step further includes systemic application of the anticholinergic quaternary amine compound as a preparation selected from the group consisting of tablets, caplets, capsules, syrups, elixirs, lozenges, suspensions, emulsions, intravenous drips, injections, mucoadhesives, and ophthalmic drops.

Usefully, the anticholinergic quaternary amine compound is charged at a physiological pH to minimize systemic absorption of the anticholinergic quaternary amine compound when localized treatment is desired.

### DESCRIPTION OF THE DRAWINGS

These and other features, aspects and advantages of the present invention will be more fully understood when considered in combination with the following specification, appended claims, and accompanying drawings, wherein:

FIG. 1 is a semi-schematic illustration of the structural differences between ammonia, primary, secondary, tertiary and quaternary amines;

FIG. 2a is a semi-schematic illustration of the structure of glycopyrrolate;

FIG. 2b is a semi-schematic illustration of the structure of a minimum pharmacophore of an anticholinergic quaternary amine;

FIG. 3 is a semi-schematic illustration of the structure of myconazole as an exemplary imidazole;

FIG. 4 is a semi-schematic illustration of the structure of ketoconozole as an exemplary triazole;

FIG. 5 is a semi-schematic illustration of the structure of an exemplary solanaceous alkaloid;

FIG. 6 is a semi-schematic illustration of the structure of an exemplary aminoalcohol ester, suitable for use in connection with the present invention;

FIG. 7 is a semi-schematic illustration of the structure of an exemplary aminoalcohol ether; and

FIG. 8 is a semi-schematic illustration of the structure of isopropamide iodine as an exemplary aminoamide.

### DETAILED DESCRIPTION OF THE PREFERRED EMBODIMENTS

The present invention is directed to compounds and methods for treating a subject or patient suffering from an infection, whether pathogenic or not, by administration of a therapeutically effective amount of an anti-cholinergic quaternary amine (ACQA) compound. The present invention is further directed to antimicrobial compounds suitable for use as pharmaceutical, cosmetic or device I compositions with end benefits that relate to clearing the microbe and possibly include deodorization. The particular systems and methods of the invention are predicated on the surprising discovery that glycopyrrolate (an ACQA, also termed GP) has anti-fungal/fungicidal as well as antibacterial effects. In particular an ACQA compound may be administered alone or in conjunction with other known anti-fungal/antibacterial agents, with the ACQA compound enhancing the effect of such other known anti-fungal/antibacterial agents. Accordingly, the administration of an ACQA reduces the amount of another anti-fungal/antibacterial agent required for effective therapy, thereby limiting the potential toxic response and/or high cost of conventional treatment. Additionally, administration of an ACQA compound may enhance or accelerate the effects of such other known anti-fungal/antibacterial agents.

Antimicrobial Activity

In Vitro and in vivo tests can be used to show the efficacy of antimicrobial products. With appropriate controls and methods In Vitro tests are applicable to all products. The time-to-kill test is used to determine the killing kinetics and activity spectrum of antimicrobials. In this example, the test was, as is usual, performed in suspension. The principle is to place the product or agent in contact with an inoculum for a specified period of time. At the end of the contact time, the mixture is inactivated by dilution into neutralizing broth. Serial dilutions in appropriate broth are performed and the number of surviving bacterial observed on solid culture media. It is general practice to test agents so that the final concentration is representative of the use concentration of the product.

In the instance of anti-fungal activity, detailed analysis has been carried out with a prototypical pathogen, Trichophytone mentagrophytes. The testing was conducted and illustrates the activity of the ACQA agents that are the subject of this invention. A Time Kill (D-Value) study (ASTM protocol #1891-97 Standard Guide For Determination Of A Survival Curve For Antimicrobial Agents Against Selected Microorganisms And Calculation Of A D-value And Concentration Coefficient) was carried out to screen for the antifungal activity of glycopyrrolate (3%). Testing was done to determine the speed at which the product kills a representative test organism, which was Trichophyton mentagrophytes, a representative dermatophyte-causing species. Determination of the minimum inhibitory concentration (MIC) versus A. Niger, a prototypical fungi was also initiated. The vehicle was 65% water/35% ethanol.

In the D Value study, the vehicle showed some reduction in organisms after 215 minutes, reflecting the known limited effect of alcohol on fungal organisms. Unexpectedly, the ACQA, glycopyrrolate (3%) delivered an antifungal effect over and above that of the vehicle, after only 24 minutes reflecting a fungicidal benefit of the ACQA. The MIC study showed activity in the range >0.1%. The data provide a clear demonstration of antimicrobial efficacy against a classically representative organism relevant and related to prevention or treatment of fungal infections. Insofar as ACQAs have a documented history of safety, albeit for other uses, these compounds are favorable alternatives to the available antifungal compounds. This prototypical ACQA agent, GP, is a drug that is already in clinical use as an anticholinergic drug, but this new use of ACQAs provides an important new addition to the limited arsenal of anti-fungal agents that can be used for medicinal purposes.

Additional scientific investigation was completed to verify the drug class aspects of this invention. Notably, two other ACQA compounds having demonstrably different chemical structures were shown to have like effects. Specifically, Mepenzolate bromide and Ipratropium bromide were evaluated utilizing standardized microbiological test methods for minimum inhibitory concentration (MIC) versus A. niger and C. albicans. MICs in the range of 1% were observable, confirming the antifungal activity of this class of compound. Insofar as ipratropium is used for intranasal purposes, it is apparent that the discovered property is not predicted by any known attribute of this class of drugs.

Similarly, In Vitro and In Vivo tests can be used to show the efficacy of antimicrobial products against bacterial species. Such a Time Kill (D-Value) study was conducted and illustrates the antibacterial activity of the ACQA agents that are the subject of this invention. In this case, the study was carried out to screen for the antibacterial activity of glycopyrrolate (3%) and triclosan (0.3%). Testing was done to determine the speed and MIC at which the product kills representative test organisms, which were Micrococcus luteous (syn ATTC 9341 changed to Kocuria rhizophila), Staphylococcos epidermidis and Corynebacterium vitaeruminis, representative odor-causing species. Once again, the vehicle was 65% water/35% ethanol.

The vehicle showed no reduction in any organisms at zero time or five minutes. The saline control showed expected recovery at zero time, 5 minutes and 30 minutes, with no reductions. Unexpectedly, the anticholinergic quaternary ammonium, glycopyrrolate (3%) delivered an immediate kill (4 minutes) in the D Value study. The MICs were between 0.1 ans 1.0%. In its effects, the ACQA was as effective as triclosan, a positive control, reference standard compound.

Further testing of the antibacterial effects of other compounds in the class of ACQA compounds was completed to verify that the observations were attributable to the class. The Minimum Inhibitory Concentrations (MICs) of Mepenzolate bromide and Ipratropium Bromide versus Staphyloccus aureus, E. Coli, and Pseudomonas were determined. The MICs were observable at 0.5-1% concentrations. The above data provide a clear demonstration of antimicrobial efficacy against the range of organisms relevant and related to prevention or treatment of problems from diverse microbial causes including malodor on mammalian skin. Insofar as ACQAs have a documented history of safety, albeit for other uses, these compounds are favorable alternatives to the irritating quaternary surfactants that have been used in the past as disinfectants.

Biologically active quaternary anticholinergic compounds (ACQAs) have been classically characterized by their ability to exert effects at the acetylcholine receptor without any notable irritation. These agents have been administered into the eye, the vasculature, the muscle and the stomach without any of the irritating properties of the disinfectant quaternary compounds. This difference in their properties clearly distinguishes these materials that happen to share the common chemical structure of a quaternary ammonium species.

Inhibiting non-pathological body odors, in accord with the present invention, involves topical application or administration of an anticholinergic composition to an effected body area. Once applied, the composition prevents or affects odor formation via a direct action upon the bacteria resident upon the skin. Alternatively, the composition can act indirectly to prevent odor formation. In this aspect, the composition penetrates the skin of the body area with the anticholinergic composition, and blocks the result of sympathetic cholinergic nerve fiber releasing acetylcholine to an innervated sweat gland with the anticholinergic composition. The blockade of the acetylcholine receptor interrupts the sweat process, resulting in localized drying, and thereby creating conditions unfavorable to the odor-causing microorganisms. In actual practice both mechanisms operate to reduce odor.

Numerous anticholinergic materials have been identified and are in clinical use or development. In general, and as described above, these can be categorized as primary, secondary, tertiary or quaternary amines. Those that are most suitable for the envisioned application reside on and/or penetrate the skin without irritation. It is an advantage if they are charged within the body so as not to easily cross biological membranes. Glycopyrrolate is an example of such a material. It is a quaternary amine that carries a positive charge at physiological pH. Like other ACQAs, glycopyrrolate is water-soluble and also overcomes the formulation limitations of triclosan.

The specific activities of the ACQA's of the present invention are unexpected and surprising given the recognized nature, uses and limitations of the selected general chemical class of the materials under discussion. Specifically, the prototypical ACQA agent, GP, is conventionally prescribed to prevent painful spasms of the gut and urinary bladder. Glycopyrrolate relaxes the muscle wall of the gut and the urinary bladder in order to prevent spasms from occurring, as well as slightly reducing the production of stomach acid. Although initially approved for use in the treatment of peptic ulcers, it is often useful in treatment of conditions such as diarrhea, irritable or spastic bowel, diverticulosis, colic and bladder spasm.

The bacterial flora of the skin is relatively simple. The axilla supports a high density of bacteria but importantly, recovery from the axilla is representative of the indigenous flora of the skin in general. The microorganisms resident on skin are the preferred targets for the treatment of body odor problems utilizing the compositions. The resident microflora, which are present at concentrations up to 10^{6/}cm² include aerobic cocci of the Micrococcocae family, lipophilic aerobic diptheroids, primarily Corynebacterium species, anaerobic diptheroids such as Proprionibacterium, yeast from the genus Pityrosporum and occasional Gram-negative species of various genera.

In the underarm, the Coryneform bacteria and micrococci predominate. These organisms are well adapted to their ecological niche, which is an environment of higher moisture, relative to most body parts. This moisture is conducive to bacterial proliferation.

Strong body odors are generally associated with the dominance of Coryneform bacteria. The Coryneform bacteria were favored in research looking at excessive hydration. It is generally thought that Corynebacterium species dominate the composition of the axillary flora although cocci and diptheroids have important ecological roles as well. The identification of the actual odoriferous molecules has been the subject of much research as well. Fatty acids, such as 3-methyl-2-hexanoic acid as well as steroid-based molecules have been targeted as causative odorants. The link between odor and bacteria results from studies that demonstrate the role of bacteria in generating odor from otherwise odorless secretions. Likewise, numerous studies now demonstrate that a decrease in axillary odor correlates to a decrease in axillary bacteria. A condition termed bromhydrosis refers to objectionable smelly sweat that interferes with people's lives.

In the context of antiseptic use, quaternary ammonium compounds (ACQAs) are generally described as surfactants. As such, they have the ability to modify the interface between otherwise incompatible lipophilic and hydrophilic materials. Quaternary amine surfactants have been used, historically, for their ability to clean which is, in fact, a function of their ability to break down and emulsify lipid-like materials. An inherent limitation on their use is the damage to lipid-based body structures such as skin. Thus, quaternary amines are used at extremely high dilutions (i.e. low concentrations) to prevent them from creating skin irritation at the same time that they are being used for cleansing benefits. Notably, the quaternary amine surfactants and disinfectants have no physiologic role. This clearly distinguishes them from the potent, biologically active quaternary compounds with anticholinergic efficacy, which are the subject of this invention.

The antimicrobial activity of the ACQAs according to the present invention is unexpected and surprising given the recognized nature, uses and limitations of the selected general chemical class of materials. Contrary to what one might expect, the subject ACQAs do not have this classical profile of irritation or pattern of use. Rather, these agents have been used clinically on delicate body structures including the nasal mucosa, lungs, and the stomach, within the vasculature and in the eye for the rather specific purpose of blocking the action of acetylcholine on its cell receptor. These very specific uses have no application to the newly discovered antimicrobial properties. For example, the use of an ACQA to inhibit gastric acid secretion does not involve a nexus or interaction with bacteria or fungi. Indeed, the bacteria against which the AQAS work to affect body malodor do not even live in the areas which are subject to pharmacological treatment when the compounds are taken orally or parenterally. It is, therefore unexpected that quaternary ammonium anticholinergic compounds (ACQAS) should exert any antibacterial or antifungal effect. However, this benefit has now been discovered.

In accordance with the present invention, ACQAs are applicable, in addition to systemic infections, to dermatomycoses which typically occur in areas such as the feet, nails, palms, scalp, genital area, back, groin and other moisturized parts of the body. One described embodiment comprises the topical application of a composition including a quaternary amine anticholinergic agent to such parts of the body as can become infected with mycoses.

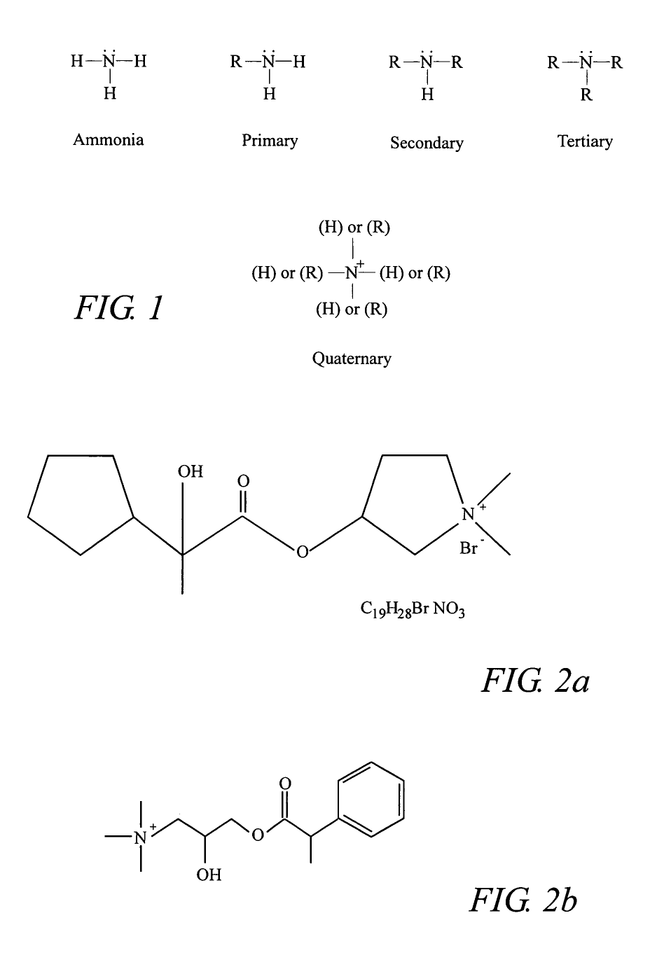

With regard now to the illustrated embodiment of FIG. 1, numerous anticholinergic materials have been identified and are in clinical use or development. In general, these can be categorized as primary, secondary, tertiary or quaternary amines. As discussed above, the adjectival description of an amine refers to how many organic groups are bonded to the nitrogen atom. As shown in the exemplary embodiment of FIG. 1, a primary amine is distinguished from ammonia by having a single organic group (an R group) replacing one of the hydrogen atoms. A secondary amine has two R groups (which may or may not be the same or similar), substituting for hydrogen; a tertiary amine likewise has three R groups substituted for the hydrogen atoms.

Those anticholinergic materials that are within the contemplation of the present invention must reside on and/or penetrate the skin without irritation. Advantageously, they should be charged within on and in the body so as not to easily cross biological membranes. Although certain tertiary amines exhibit this feature (if protonated), quaternary amine molecules are inherently ionic (charged) in character.

As exemplified in FIG. 2a, glycopyrrolate is an example of such a material. Glycopyrrolate (also termed herein as GP) is a quaternary amine that carries a positive charge at physiological pH. Glycopyrrolate is an ionic compound, and is depicted, for exemplary purposes only in the embodiment of FIG. 2a, as a bromine salt (glycopyrrolate bromide). When hydrated, GP ions have four organic groups bonded to the nitrogen. Accordingly, the nitrogen atom is not bonded to a hydrogen which might be able to be removed by a base (thereby resulting in an acid). In the absence of a possible acid-base reaction, the GP structure is unaffected by changes in pH.

Although glycopyrrolate is discussed in terms of an exemplary ACQA, certain features of ACQAs, particularly those having anticholinergic activity, are shared, as depicted in the exemplary embodiment of FIG. 2b. In terms of anticholinergics, the structure-activity relationships for these compounds depend on inclusion of certain general structural features. A charged nitrogen moiety (a cationic head) allows the quaternary ammonium group to attach to acetylcholine receptors (in the case of anticholinergic compounds). Although not required for activity, compounds that possess a hydroxyl substituent are generally more potent than the corresponding non-hydroxylated analogue. The most potent anticholinergic agents possess an ester functionality that mimics the ester group in ACH, and at least one bulky cyclic substituent (phenyl, thienyl, cyclohexyl, etc.) is present in almost all anticholinergic agents.

The inventors make no claim that the ACQAs of the present invention must include these structural features. Indeed, other compounds were shown to demonstrate the invention have markedly different chemically structures. However, it is noted that GP includes all such structure and has demonstrated antifungal activity.

The effective use of ACQA's, therefore, requires that a sufficient amount of ACQA be applied to the effected area of the skin or otherwise administered to reach the target orgasms at an effective concentration. An ACQA compound or derivative compound, in accordance with the present invention, may be administered systemically or topically. Systemic routes of administration include oral, intravenous, intramuscular intranasal or subcutaneous injection, intraocular or retrobulbar, intrathecal, intraperitoneal (e.g., by intraperitoneal lavage), transpulmonary (using aerosolized or nebulized forms of the drug), or transdermal. Topical administration routes include the skin and mucosa, wherein administration may be in the form of ointments, gels, salves, wraps, films, eye drops, intranasal and sinus sprays, ear drops, or irrigation fluids, such as those used for the irrigation of wounds or trauma.

Administration of ACQA compounds is particularly beneficial in immunocompromised patients, including immunosuppressed and neutropenic patients, such as persons undergoing chemotherapy, radiation therapy, or immunosuppressive therapy. ACQA compounds are further suitable for patients with a dysfunctional immune system secondary to infection, such as HIV infection or other similar causes. Topical administration of derivative compounds of the invention are also considered to be effective for treating a variety of primary fungal infections, including skin and eye infections and those caused by dermatophytes. Advantageously, the percutanous absorption of ACQA may be accelerated or improved by enhancers of microbial penetration or skin substantivity.

Pharmaceutical, cosmetic or device compositions, in accordance with the invention, suitably include an ACQA compound with anti-microbial properties and an acceptable diluent, adjuvant or carrier for administration via the selected route e.g. topical, intravenous or oral. The product may be presented as an aerosol or spray or other appropriate dosage form. The ACQA compound may be administered in conjunction with known surfactants, other chemotherapeutic agents or in combination with additional known anti-microbial agents.

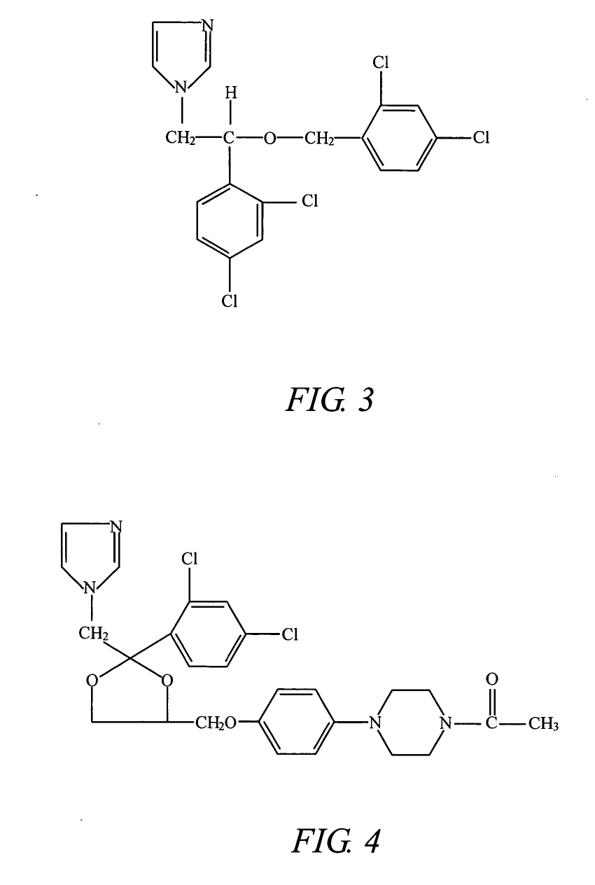

Certain non-ACQA anti-fungal agents that are useful in connection with the present invention include azole derivatives such as the imidazoles and triazoles. Azole derivatives are well known anti-fungal agents, certain of which inhibit cytochrome P450 dependent enzymes (particularly C14-dimethylase) involved in the biosynthesis of ergosterol, which is in turn required for fungal cell membrane structure and function. FIGS. 3 and 4 illustrate, respectively, the structures of myconazole and ketoconazole, which are representative of triazoles and imidazoles having antifungal properties.

Other non-ACQA antifungal agents include allylamine and morpholine anti-fungal drugs that inhibit ergosterol biosynthesis at the level of squalene epoxidase (or the same pathway at a later step). Polyene anti-fungal drugs, such as amphotericin, nystatin, and primaricin, interact with sterols in the cell membrane (ergosterol in fungi, cholesterol in humans) to form channels through which small molecules leak from the inside of the fungal cell to the outside. Additionally, antimetabolite anti-fungal drugs, such as 5-fluorocyctosine, act as an inhibitor of both DNA and RNA synthesis via the intracytoplasmic conversion of 5-fluorocyctosine to 5-fluorouracil.

Concurrent administration of an ACQA compound, in accordance with the present invention, with other complimentary agents, is expected to improve the therapeutic effectiveness of the complimentary anti-fungal agents. Improved effectiveness is characterized by the reduction of the concentration of the anti-fungal agent required to eradicate or inhibit fungal growth or replication. Because the use of certain anti-fungal agents is limited by their systemic toxicity or prohibitive cost, lowering the concentration of anti-fungal agent required for therapeutic effectiveness reduces this toxicity, as well as reducing the cost of treatment, thereby allowing for a more widespread use of the agent. Concurrent administration may also expand the spectrum of anitmicrobial utility while maintaining safety. Concurrent administration of an accompanying anti-fungal compound, in accordance with the invention, provides for a more rapid and/or complete anti-fungal/fungicidal effect than could be achieved with either agent alone. Administration of an ACQA compound will also reverse the resistance of fungi to anti-fungal agents. Administration of an ACQA compound, either alone or in combination, will also convert a fungistatic agent into a fungicidal agent.

Concurrent administration, as the term is used herein, includes administration of the agent either simultaneously, or before, or after one another, by any combination of medically acceptable routes of administration. For example, an ACQA compound may be administered intravenously while the other anti-fungal agents are administered topically, intramuscularly, intravenously, subcutaneously, orally or intraperitoneally. An ACQA compound, along with concurrently administered anti-fungal agents, may be administered simultaneously or sequentially, so long as they are given in a manner sufficient to allow both agents to achieve effective concentrations at the sight of infection.

One particular example, not intended to be limiting, of a complimentary combination is an ACQA provided in combination with a moisture blocking agent, such as an antiperspirant. It is well known that fungi often flourish in warm, moist conditions, particularly where they have easy access to nourishment such as skin secretions and skin cell debris. Prevention and treatment of fungal infections might include efforts to deprive the organisms responsible for the infection of the moist/humid environment that they require in order to proliferate and grow. Accordingly, fungal infections may be controlled by moisture absorption agents, as well as antiperspirant agents that function to suppress the manufacture of sweat or block the expression of sweat, generally eccrine in nature, onto the skin surface. Because organisms that generally produce fungal infections live optimally in the presence of moisture, the reduction of surface moisture by virtue of reduced perspiration output may carry indirect anti-fungal benefits. In this regard, effective dosages, combinations and administration regimens, for compositions including an ACQA compound in combination with a moisture absorption or moisture suppressing agent can be readily optimized by one having skill in the art, as determined by good medical practice and the clinical condition of the individual subject.

Another benefit of the ACQA-antiperspirant combination is in the condition of bromhydrosis. Here, the lack of surface moisture will impede the growth of organisms that feed on apocrine sweat to produce malodorous materials. The AQAC will directly impact those microbes, including the corynabacterium and diptheroids and that benefit will be further enhanced by the dryness that is hostile to the organisms.

In particular, it would be appreciated that certain ACQA compound embodiments may be incorporated with a metal salt or other antiperspirant agent in order to provide a synergistic combination. Metal salt antiperspiration compositions may include such antiperspirant agents as aluminum salts, zirconium salts, mixed aluminum/zirconium salts, and the like. Anti-fungal activity is accomplished by both a direct fungistatic/fungicidal effect on the organisms, as well as accomplished by changing the microenvironment to be hostile to the organisms. In such situations, it will be understood that a lower concentration of anticholinergic compositions may be employed. It should further be noted that compounds with anticholinergic effects not only diminish the effect of acetylcholine, but also themselves inhibit the production of saliva, sweat and bronchial secretions.

An ACQA anti-fungal compound may also be administered in combination with an anti-bacterial agent or anti-inflammatory agent in such situations as the treatment of sinusitis or a topical skin aliment. An anti-bacterial agent assists in preventing secondary infection and may indeed supplement the primary anti-fungal affect with respect to the range of organisms that might simultaneously reside on the skin (i.e., eliminate or reduce odor causing bacteria that may accompany the primary fungal problem). An anti-inflammatory agent is particularly suitable in reducing undesirable symptomatic inflammations, as well as promoting healing.

Importantly, therapeutic effectiveness is based on a successful clinical outcome and does not require that the anti-fungal agent or agents eradicate 100% of the organisms involved in the infection. Success depends on achieving a level of anti-fungal activity at the site of infection that is sufficient to inhibit the fungi in a manner that tips the balance in favor of the host. When host defenses are maximally effective, the anti-fungal effect required may be minimal. Reducing organism load, at the affected area, by even one log (1 order of magnitude or a factor of 10) may permit the host's own defenses to control the infection. In addition, augmenting an early fungicidal/fungistatic effect can be more important than providing a long-term fungicidal/fungistatic effect. These early events are a significant and critical part of therapeutic success because they allow time for host defense mechanisms to activate.

For any of the antimicrobial uses, Glycopyrrolate (GP) or any other ACQA compound may be utilized in a simple solution or in an emulsion, dispersion, suspension, or as a liquid crystal, creme, gel or ointment base. The agent may be used neat or encapsulated partially or in entirety (e.g., a clathrate, cyclodextrin, liposome or other coated particle) or in combination in order to control solubility, release rate or dose time profile. If incorporated in a patch or film, the anticholinergic may be formulated to be delivered on the matrix structure, within the matrix structure or alternatively, as part of an adhesive system in a manner generally similar to the well known scopolamine anti-seasickness patch. Additionally, glycopyrrolate might be administered via a system that utilizes a membrane, either physically or chemically based, to control the delivery of the drug. Whichever administration method is chosen, given its chemical state, and the related resistance to crossing biological membranes, it is unexpected that ACQAs would be sufficiently effective to treat systemic infection via a transdermal route of administration, unless some mechanism for penetration enhancement is built into the dosage system.

In accordance with the invention, amines having pKa's higher than about 9.0 or 9.5 are preferred to ensure that substantially all of the molecules are ionized at normal physiological pH. Molecules that are charged at physiological pH are essentially unable to pass cell membranes such that topical administration is unlikely to result in systemic effects. This does not, however, preclude systemic use. It will be appreciated by those having skill in the art, that an ACQA, such as glycopyrrolate, which is hydrophilic and charged at physiological pH (inherently poorly absorbed) is unexpectedly affective at accomplishing anti-microbial effects.

It is moreover unexpected that effectiveness is observed at concentrations of only about 3 percent and without irritation known to characterize the quaternary ammonium compounds previously identified as having antimicrobial efficacy.

The effective use of ACQAs, therefore, requires that a sufficient amount of ACQA be applied to the skin to reach the organisms at an effective concentration. This treatment provides a topical antimicrobial deodorant. Though not necessary to the preferred embodiments, activity of the ACQA may be enhanced by enhancers of microbial penetration or of skin substantivity.

In a manner similar to the above-described fungistatic/fungicidal instance, the product formulation is dependent upon what is appropriate or desired for the form. It may be opaque, translucent or clear. It may be dry, powdered, liquid, a loose gel, a formed, structured stick, semisolid or a soft solid, a patch or an adhesive film, either meltaway or substantive, semiocclusive or occlusive. It also may be anhydrous or water based, utilizing such combination of components as provides the desired profile of dose and aesthetics. In general, it will include a vehicle/carrier that can comprise dispersants, emollients, fragrance, surfactants and structurants.

The formulation can include a base such as stearic acid or sodium stearate, water, wax and/or silicone fluid; it may include other structural agents including, for example, gums, silica, polymer systems or gellants and at least one anticholinergic drug at concentrations ranging from 0.0001% to 20% w/w, with a preferred range of 0.001% to 10% and a more preferred range of 0.01% to 10%. The active ingredient may be a free base, salt or analogue of the drug. It may be in a racemic or chirally purified form. The term glycopyrrolate as used herein is intended to be broader than the compound of that name unless indicated otherwise; it is a quaternary ammonium compound that also includes analogues wherein the chemical structure has been modified to introduce, modify or remove or change functionalities of the structure. For example, such modification can result in the removal of an OH functional group and the like. Insofar as the modified molecule is antimicrobial, it is likewise within the contemplation of the invention. By virtue of the presence of a quaternary amino group, compounds of this invention readily form salts. The drug is acceptable with a counter salt.

When used for either purpose, product formulation is necessarily dependent upon what might be appropriate or desired for the form of use. It might be opaque, translucent or clear; dry, powdered, liquid, a loose gel, formed stick or tablet, semi-solid or a soft-solid, a patch or adhesive film, either melt-away or substantive, semi-occlusive or occlusive. It might also be anhydrous or water based, utilizing such combination of components as provides the desired profile of dose and aesthetics. In general, the product formulation will include a vehicle or carrier and might also include a dispersant, emollient, surfactant, structurant, viscosity control agent, foaming agents or even a fragrance.

Although numerous anticholinergic compounds are usable in connection with the present invention, ACQA's such as glycopyrrolate, are inherently water soluble because of their ionic nature and, therefore, may be either suspended in a hydrophobic milieu, such as a hydrocarbon, silicone or wax based matrix or system, in an oil emulsion, water emulsion, or dissolved in a hydrophilic or aqueous milieu either as a solution or gel. End products can come in a variety of forms and matrices. The topical application provided by such forms preferably occurs in an open system, namely, where patches are omitted and the ACQA is active while the treated area is exposed to temperatures, light and air. In the form of a shampoo, water might be present.

Active ingredients may also be incorporated into a reservoir or other type of “patch” or plaster, or embedded in a matrix for controlled release. For example, a sole insert, or a sock or glove, or even wrapping tape might be utilized to deliver an agent to requisite areas of a foot or palm. Such systems have an added advantage of focusing delivery to a desired portion of the body surface while protecting other surface portions from unintentional or undesirable exposure. Active agents may be delivered in a film forming matrix, in which case the film layers develops once the product is applied. Such films may create occlusion in order to enhance penetration or may simply create a reservoir of the drug in a localized region of the skin. Alternatively, the active agent may be contained in a system that incorporates a membrane, or other such structure in order to control the timing and/or concentration of drug release.

In summary, a product formulation may include materials which functionally serve as structurants and/or structure enhancers or modifiers, emollients, surfactants, co-surfactants, fragrances, emulsifiers, glidants, dispersants, disintegration aids, tableting aids, suspending agents, wash-off agents, controlled release agents, and penetration enhancing, controlling or release agents. Materials within a given functional group may also be combined in order to balance a multiple set of benefits. For example, low and high melting point waxes may be combined in order to optimize manufacturability or attributes of use. Nonwoven or woven backings or even cellophane might be utilized in an application matrix. Likewise, polyethylene waxes or triglycerides may be used alone or in combination with classic organic materials. These components could be characterized as polymers or derivatives of polymers with optimized functionality. Similarly, silicone and silicone derivatives might be combined to control and vary product properties. Product formulations might also include materials with properties that are keratolytic, astringent, anti-oxidant, anti-inflammatory, antihistaminic, antiperspirant, anti-bacterial, or growth enhancing, and/or materials promoting or retarding penetration, moisturization, lubrication, barrier protection, wound healing, cellular turnover, cellular enzyme metabolism, modulating growth factors, providing UV protection, depilation, hair growth retardation, skin or hair conditioning, and the like. Other included materials might exhibit activities that are intended to impart benefit to the skin itself or enhance the functionality of the formulation or its application. Depending on the application, skin-softening agents such as urea might further be included, for example, in a foot care product.

For use as an antibacterial deodorant, the vehicle may comprise an anhydrous solution, a suspension of the amine in a hydrophobic matrix, water, alcohol, a hydroalcoholic solution or other acceptable carrier material. It can be dry, as in a powder or powder blend. For example, if the product is to be used as a dry powder, carrier powders can be included to enhance ease of dispensing, application or skin substantivity. Sodium bicarbonate, talc and starch are examples of dry powder adjuncts that can be used. The composition can further comprise a fragrance, an anti-perspirant, an anti-fungal agent, a skin penetration enhancer, and/or an emulsifier, a hair growth retardant or inhibitor or a skin or hair conditioner. In any case, the anticholinergic amine preferably has a pKa greater than 9.0 and the anticholinergic amine is charged at a physiologic pH.

Topical preparation containing the active compound can be admixed with a variety of carrier materials or pharmaceutically acceptable excipients well known in the art. Where the excipient serves as a diluent, it can be a solid, semi-solid, or liquid, which acts as a vehicle, carrier or medium for the active ingredient. Thus, the compositions can be in the form of powders, suspensions, emulsions, solutions, syrups, alcoholic solutions, ointments, topical cleansers, cleansing creams, skin gels, skin lotions, mousses, roll-ons, aerosol or non-aerosol sprays in cream or gel formulations and soft gelatin capsules.

Some examples of suitable excipients include alcohols, aloe Vera gel, allantoin, glycerin, vitamin A and E oils, mineral oil, PPG2, myristyl propionate lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, glycerin, syrup, and methyl cellulose. Others may include hyaluronic acid, its derivatives or other mucopolysaccharides. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring or fragrance agents. The compositions of the invention can be formulated so as to provide quick, sustained or delayed release of the active ingredient after administration to the patient by employing procedures known in the art.

The compounds of the present invention may be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

It should be understood that a product formulation might also contain a skin foundation, color cosmetic, toning agent, or the like. Certainly, it might also include, in combination, an additional anti-fungal agent of a differing chemical class, or a metal salt antiperspirant/deodorant, in order to enhance combined efficacy via different mechanisms of action.

Further, U.S. Patent Application Publication No. 2002/0037264 entitled “ANTIPERSPIRANT AND DEODORANT PRODUCTS AND METHODS FOR THEIR USE,” describes additional materials and components that are suitable for incorporation in an antibacterial/antiperspirant product in accord with the present invention. Accordingly, the entire contents of U.S. Patent Application Publication No. 2002/0037264 are expressly incorporated herein by reference. For example, and not by way of limitation, metal salt antiperspiration compositions may include those disclosed in the '264 application, such as aluminum, zirconium, mixed aluminum/zirconium salts, and more. Antiperspirancy is thus accomplished by both interruption of the conduction of the homeostatic nerve signal as well as blocking of skin pores. In such cases, a lower concentration of the preferred anticholinergic compositions may be employed.

In light of the possible potency of the compounds of the invention, the package may be used to help control the delivery of a proscribed amount of the composition. For example, it can be in a dispenser providing an audible or palpable click representing the advancement of a specific volume of product through a dispensing mechanism. Alternatively, the product can be formulated or packaged in a unit dose format or a swab or brush on applications might be envisioned.

The composition of the invention may be formulated so as to provide quick, sustained or delayed release of an active ingredient after administration to the patient by employing various procedures well known to those having skill in the art. The formula may also contain other materials with natural origin with complementary biological activity, such as antioxidant, wound healing or anti-microbial properties including peptides. The composition may additionally include one or more optional additives such as colorants, disintegrants, perfumes, and the like. In practice, each of the additional additive materials should be both miscible and compatible with the ACQA compound. In this regard, compatible additives are merely those that do not prevent or inhibit the use of an ACQA compound as an antibacterial/anti-fungal agent, in the manner described herein.

Specifically, the product formulation includes a base and at least one anticholinergic drug present at a concentration ranging from about 0.0001% to about 20% w/w, with a preferred range from about 0.001% to about 10% w/w and more preferably, in the range of from about 0.001% to about 5% w/w. The active ingredient is provided as a free base, salt or analogue of the drug. The active ingredient is a quaternary ammonium compound that might also include analogues, wherein the chemical structure has been modified to introduce, modify or remove, or change functionalities of the structure. For example, and in the case of glycopyrrolate, such modification can result in the removal of an OH functional group, or the like. Insofar as the modified molecule is bacteriostatic, bactericidal, fungistatic or fungicidal, such modified molecules are encompassed within the scope and spirit of the present invention. By virtue of the presence of a quaternary amino group, compounds of the invention readily form salts, and the drug is acceptable with a counter salt. An anticholinergic amine preferably has a pKa greater than 9.0, and is charged at a physiologic pH.

In preparing a formulation, the active compound may be milled in order to provide an appropriate particle size prior to combining the active ingredient with other, optional, ingredients. Where the active compound is substantially insoluble, it is typically milled to a particle size of less than 200 mesh, corresponding to a particle size of about 75 microns, in accordance with the well known ASTM sieve designation. It may also be processed so as to be present in the nanometer size. Where the active compound is substantially water soluble, particle size is normally adjusted by milling to provide a substantially uniform distribution in the formulation. Typically, it is milled to a particle size of about 40 mesh, corresponding to approximately 425 microns, under the ASTM sieve designation.

ACQA's are generally hydrophilic, however the onset of local perspirancy does not necessarily void activity. Conversely, an ACQA, such as glycopyrrolate, is provided in a certain hydrophobic form or matrix, in order to provide a desirable delayed response. Such a timed-release response occurs when local perspirancy first diffuses into the hydrophobic matrix (that might be present as a film on the skin's surface), which in turn causes the ACQA to be dissolved such that it is able to diffuse to and suppress the activity of the causative microorganisms.

Although the exemplary embodiments of the invention have been discussed in connection with a particular ACQA, glycopyrrolate, it should be expressly understood that a large number of ACQA's are effective and may be employed in connection with the present invention. Such ACQA's include, but are certainly not meant to be limited to, methscopolamine, homatropine, methantheline, propantheline, ambutonium, benzilonium, dibutoline, diphemanil, emepronium, blycopyrronium, isopropamide, lachesine, mepenzolate, methantheline, oxyphenonium, propantheline, ipratropium, n-methyl atropine, n-methylhyoscine, methobromide, and similar anticholinergic amines. Indeed, glycopyrrolate may be substituted with any single ACQA, or any combination of ACQA's, which exhibit properties for controlling eccrine sweat at concentrations that do not cause serious physiological side effects. These required concentrations will vary, given the varying but consistently high potency of these materials in general, but variation calculation is easily within the scope of one having ordinary skill in the art and, as such, are not specific to practice of the present invention. Nevertheless, in the case of ACQA's, or even agents that are not ACQA's, it is important to select a particular compound with a pKa indicating that the compound will be substantially entirely charged at physiological pH in order to prevent the composition from being absorbed systematically, when localized treatment is the objective.

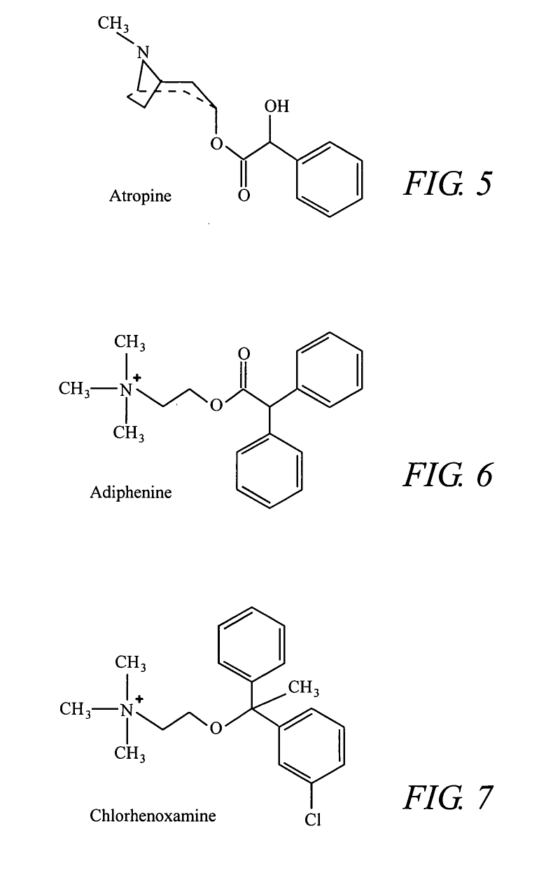

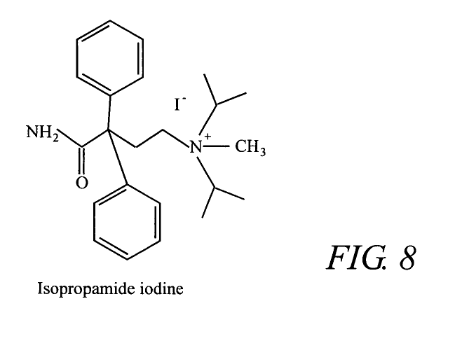

Further, is will be understood that the ACQAs, in accord with the invention, include not only naturally occurring anticholinergic compounds or agents, but also synthetic anticholinergic compounds and agents. In addition to anticholinergics for which glycopyrrolate might serve as a prototype, the general class of ACQAs include solanaceous alkaloids, an exemplary embodiment of which is depicted in FIG. 5, as well as various aminoalcohol esters and aminoalcohol ethers which exhibit anticholinergic activity. A selected exemplary embodiment of an aminoalcohol ester and an aminoalcohol ether is depicted in FIGS. 6 and 7 respectively. Aminoamides are similar in electronic character to the aminoalcohols, since a polar amide group replaces the alcohol functionality as a surrogate for the aminoester. Accordingly, these classes of compounds are within the contemplation of the present invention. A representative aminoamide is isopropamide iodine as depicted in FIG. 8.

Quaternary amine compounds may be presented as a salt or may, for convenience or specific intent, be delivered as a derivative. Acceptable countersalts of quaternary amines can be prepared from inorganic and organic acids. By way of example, these may include but are necessarily limited to hydrochloric, hydrobromic, sulfuric, nitric, phosphoric, glycolic, pyruvic, oxalic, malic, malonic, succinic, maleic, fumaric, tartaric, citric, benzoic, cinnamic, mandelic, methanesulfonic, ethanesulfonic, p-toluene-sulfonic, salicylic acids as well as hydrogen fluoride, hydrogen iodide, and the like. The counter ion chosen can be important for producing a solution that is near to a neutral pH. The counter ion may also compliment the ACQA with respect to antimicrobial activity as for example zinc or a chelating agent that interferes with bacterial/fungal viability.

Among those derivatives commonly known to those having skill in the art, include the so-called soft-anticholinergic drugs, and embodied herein are esters as well as synthetic analogues thereof. The range of compounds is illustrated by, but certainly not limited to, U.S. Pat. Nos. 5,618,826, 5,258,388, 5,223,528, 5,637,601, 5,418,244, 5,610,188, 5,418,244, 4,546,096, 4,517,176, 4,720,494, 3,312,709 and 3,326,768, the entire contents of which are expressly incorporated herein by reference. Bodor (Bodor, Chemtech, Jan. 28, 1984, pp. 28-38, and Bodor, in Design of Biopharmaceutical Properties Through Prodrugs and Analogs, ed. E. B. Roche, Washington, D.C., Academy of Pharmaceutical Sciences, 1977, pp. 98-135) also define principles by which one is able to design and synthesize anticholinergic agents and other pharmacologically active classes of drugs. Previous soft ester analogs of anticholinergics were synthesized from cyclopentylphenylacetic acid, phenylacetic acid and branched aliphatic carboxylic acids. These soft drugs, also known as soft analogs, are described in U.K. Patent Application No. 7848850, which is also expressly incorporated herein by reference.

Nevertheless, it would be desirable to design yet other soft anticholinergic drugs, especially to design such drugs using the “inactive metabolite approach.” According to this approach, an inactive metabolite (which could be hypothetical) is selected and reactivated by synthesizing an agent resembling the parent drug. The soft drug is designed with a molecular soft spot, which should predictably metabolize back to the inactive starting metabolite in vivo in one step and without going through toxic intermediates. The specific molecular design will be dependent upon the species as, for example, canine versus human. Although intended for a different purpose, the aforementioned strategies for designing quaternary ammonium anticholinergic compounds are included within the scope of the present invention. It is the intent that these be incorporated herein in their entirety as exemplary, and not limiting, embodiments.

Although the present invention has been discussed in connection with certain active ingredients, formulations, and the like, it will be understood that many alterations and modifications may be made by those having skill in the art without departing from the spirit and scope of the present invention. Therefore, it must be understood that the illustrated embodiments have been set forth only for the purpose of example and should not be taken as limiting the invention. It must be expressly understood that the invention encompasses other combinations of fewer, more or different ones of the disclosed elements. It should further be understood that the particular terminology used to describe the invention and its various exemplary embodiments are to be understood not only in terms of their commonly accepted meanings, but also as including generic structures, materials, or acts of which they might represent a single species.

The definitions of the words or elements defined in the specification do not only include a combination of elements which are literally set forth, but include an equivalent substitution of two or more elements for any one of the elements, or that a single element may be substituted for two or more elements. Accordingly, the scope of the inventions is to be viewed only in accordance with the appended claims.

What is claimed is:

1. A method for treating a microbial infection comprising the steps of preparing a therapeutically effective amount of an anticholinergic quaternary amine compound and administering said anticholinergic quaternary amine compound to an area of a human body exhibiting said microbial infection.

2. The method according to claim 1, wherein the administration step comprises contacting a microbe residing on or within the infected area with said anticholinergic quaternary amine compound.

3. The method according to claim 2, further comprising administration of the anticholinergic quaternary amine (ACQA) compound as a formulation in conjunction with a non-ACQA anti-microbial agent having a recommended concentration defining an effective therapeutic dose, wherein the recommended concentration of the non-ACQA anti-microbial agent is substantially reduced without reduction in effective therapeutic effect.

4. The method according to claim 2, further comprising administration of the anticholinergic quaternary amine (ACQA) compound as a formulation in conjunction with a non physiologically active base or support material, wherein the concentration of the anticholinergic quaternary amine compound in the formulation is in an amount of from about 0.0001% to about 20% w/w.

5. The method according to claim 4, wherein the concentration of the anticholinergic quaternary amine compound in the formulation is in an amount of from about 0.001% to about 10% w/w.

6. The method according to claim 5, wherein the concentration of the anticholinergic quaternary amine compound in the formulation is in an amount of from about 0.001% to about 5% w/w.

7. The method according to claim 2, wherein the anticholinergic quaternary amine compound comprises glycopyrrolate, mepenzolate or ipratropium.

8. The method according to claim 3, wherein the non-ACQA anti-microbial I agent comprises an imidazole or triazole compound, triclosan, triclocarbon, farnesol, an aluminum or zirconium salt or complex thereof, antiseptics, essential oils or other naturally or synthetically derived antimicrobial materials.

9. The method according to claim 4, wherein the non physiologically active base or support material includes additional components having non-fungicidal activities, the additional components selected from the group consisting of keratolytics, wound healing agents, antioxidants, anti-inflammatories, antihistamines, antiperspirants, anti-bacterials, moisturizers, astringents, soothing agents, lubricants, UV-protectants, depilatories, skin or hair conditioners, and hair growth retardants or enhancers.

10. The method according to claim 1, wherein the administration step includes topical application of the anticholinergic quaternary amine compound as a preparation selected from the group consisting of patches, films, sticks, gels, aerosols, non-aerosols, sprays, creams, ointments, lotions, mousses, powders, soft solids, and roll-ons.

11. The method according to claim 1, wherein the administration step includes systemic application of the anticholinergic quaternary amine compound as a preparation selected from the group consisting of tablets, caplets, capsules, syrups, suspensions, films, emulsions, intravenous drips, injections, mucoadhesives, and ophthalmic drops.

12. The method according to claim 2, wherein the anticholinergic quaternary amine compound is charged at a physiological pH to minimize systemic absorption of the anticholinergic quaternary amine compound when localized treatment is desired.

13. The method according to claim 12, wherein therapeutic effect is demonstrated by inhibiting growth of a fungal or bacterial organism responsible for the infection.

14. The method according to claim 12, wherein therapeutic effect is demonstrated by killing a bacterial organism responsible for the infection.

15. An antimicrobial composition comprising:
an excipient material suitable for topical or systemic administration; and
a quaternary amine compound having anticholinergic activity.

16. The antimicrobial composition according to claim 15, wherein the concentration of the anticholinergic quaternary amine compound in the composition is in an amount of from about 0.0001% to about 20% w/w.

17. The antimicrobial composition according to claim 16, wherein the concentration of the anticholinergic quaternary amine compound in the composition is in an amount of from about 0.001% to about 10% w/w.

18. The antimicrobial composition according to claim 17, wherein the concentration of the anticholinergic quaternary amine compound in the composition is in an amount of from about 0.001% to about 5% w/w.

19. The antimicrobial composition according to claim 16, further comprising:
a non-ACQA antimicrobial agent having a recommended concentration defining an effective therapeutic dose; and
wherein the recommended concentration of the non-ACQA agent is substantially reduced without reduction in effective therapeutic effect.

20. The antimicrobial composition according to claim 16, wherein the anticholinergic quaternary amine compound comprises glycopyrrolate, mepenzolate or ipratropium.

21. The antimicrobial composition according to claim 19, wherein the non-ACQA I agent comprises an imidazole or triazole compound or XXXX (see above).

22. The antimicrobial composition according to claim 15, the excipient comprising a material selected from the group consisting of . . . see above list keratolytics, wound healing agents, antioxidants, anti-inflammatories, antihistamines, antiperspirants, anti-bacterials, moisturizers, lubricants, UV-protectants, depilatories, skin or hair conditioners, and hair growth retardants or enhancers.

23. A method for inhibiting non-pathological body malodor comprising the steps of preparing a therapeutically effective amount of an anticholinergic quaternary amine compound and administering said anticholinergic quaternary amine compound to an area of a human body so as to act on bacteria resident on said area.

24. The method according to claim 23, wherein the administration step comprises topical application so as to contact a bacteria residing on or within the desired area with said anticholinergic quaternary amine compound.

25. The method according to claim 23, wherein the administration step further includes penetration of the skin with the anticholinergic quaternary amine compound, thereby blocking the result of sympathetic cholinergic nerve fiber releasing acetylcholine to an innervated sweat gland.

26. The method according to claim 24, further comprising administration of the anticholinergic quaternary amine (ACQA) compound as a formulation in conjunction with a non physiologically active base, support or excipient material, wherein the concentration of the anticholinergic quaternary amine compound in the formulation is in an amount of from about 0.0001% to about 20% w/w.

27. The method according to claim 26, wherein the concentration of the anticholinergic quaternary amine compound in the formulation is in an amount of from about 0.001% to about 10% w/w.

28. The method according to claim 27, wherein the concentration of the anticholinergic quaternary amine compound in the formulation is in an amount of from about 0.001% to about 5% w/w.

29. The method according to claim 24, wherein the anticholinergic quaternary amine compound comprises glycopyrrolate.

30. The method according to claim 25, wherein the anticholinergic quaternary amine compound is a charged species at physiological pH so as to minimize systemic absorption.

31. The method according to claim 26, wherein the non physiologically active base, support or excipient material includes additional components selected from the group consisting of keratolytics, wound healing agents, antioxidants, anti-inflammatories, antihistamines, antiperspirants, anti-bacterials, moisturizers, lubricants, UV-protectants, depilatories, skin or hair conditioners, and hair growth retardants or enhancers.

32. The method according to claim 23, wherein the administration step includes topical application of the anticholinergic quaternary amine compound as a preparation selected from the group consisting of patches, sticks, gels, aerosols, non-aerosols, sprays, creams, ointments, lotions, mousses, powders, soft solids, and roll-ons.

33. The method according to claim 30, wherein minimized systemic absorption promotes delayed penetration until the occurrence of local perspiration.

34. The method according to claim 24, further comprising:
providing a metal salt antiperspirant;
combining the anticholinergic quaternary amine compound with the metal salt antiperspirant; and
administering the combination to a desired area of the human body.

35. The method according to claim 34, wherein therapeutic effect is demonstrated by inhibiting growth of a bacterial microorganism responsible for the malodor.

36. The method according to claim 34, wherein therapeutic effect is demonstrated by killing a bacterial microorganism responsible for the malodor.

37. An antibacterial composition for inhibiting non-pathological body malodor comprising:
an excipient material suitable for administration of the composition to a desired area on or within the body; and
a quaternary amine compound having anticholinergic activity.

38. The antibacterial composition according to claim 37, wherein the concentration of the anticholinergic quaternary amine compound in the composition is in an amount of from about 0.0001% to about 20% w/w.

39. The antibacterial composition according to claim 38, wherein the concentration of the anticholinergic quaternary amine compound in the composition is in an amount of from about 0.001% to about 10% w/w.

40. The antibacterial composition according to claim 39, wherein the concentration of the anticholinergic quaternary amine compound in the composition is in an amount of from about 0.001% to about 5% w/w.

41. The antibacterial composition according to claim 40, wherein the concentration of the anticholinergic quaternary amine compound in the composition is in an amount of from about 0.05% to about 5% w/w.

42. The antibacterial composition according to claim 38, wherein the anticholinergic quaternary amine compound comprises glycopyrrolate.

43. The antibacterial composition according to claim 38 further comprising a metal salt antiperspirant in combination with the anticholinergic quaternary amine compound.

44. The antibacterial composition according to claim 38, the excipient comprising a material selected from the group consisting of keratolytics, wound healing agents, antioxidants, anti-inflammatories, antihistamines, antiperspirants, anti-bacterials, moisturizers, lubricants, UV-protectants, depilatories, skin or hair conditioners, and hair growth retardants or enhancers.

45. A method for killing or inhibiting the growth of microorganisms responsible for fungal infection and non-pathological body malodor comprising the steps of preparing a therapeutically effective amount of an anticholinergic quaternary amine compound and administering said anticholinergic quaternary amine compound to an area of a human body so as to counteract the effects of said microorganisms resident on or within said area.

46. The method according to claim 45, further comprising administration of the anticholinergic quaternary amine (ACQA) compound as a formulation in conjunction with an excipient, base or support material, wherein the concentration of the anticholinergic quaternary amine compound in the formulation is in an amount of from about 0.0001% to about 20% w/w.

47. The method according to claim 46, wherein the concentration of the anticholinergic quaternary amine compound in the formulation is in an amount of from about 0.001% to about 10% w/w.

48. The method according to claim 47, wherein the concentration of the anticholinergic quaternary amine compound in the formulation is in an amount of from about 0.05% to about 5% w/w.

49. The method according to claim 46, wherein the anticholinergic quaternary amine compound comprises glycopyrrolate.
